(19) United States
(12) Patent Application Publication
(10) Pub. No.: US 2024/0066162 A1
(43) Pub. Date: Feb. 29, 2024

(54) A NONWOVEN DECONTAMINATION WIPE COMPRISING A SMALL DIAMETER FIBER

(71) Applicant: 3M INNOVATIVE PROPERTIES COMPANY, St. Paul, MN (US)

(72) Inventors: Andrew W. Vail, Bayport, MN (US); Saurabh Batra, Minneapolis, MN (US); Jodi L. Connell, St. Paul, MN (US); Robert A. Schmitz, St. Paul, MN (US)

(21) Appl. No.: 18/259,602

(22) PCT Filed: Feb. 15, 2022
(86) PCT No.: PCT/IB2022/051338

§ 371 Date: Jun. 28, 2023

Related U.S. Application Data
(60) Provisional application No. 63/161,605, filed on Mar. 16, 2021.

(51) Int. Cl. A61L 2/18; A01N 25/10; A01N 25/34; A47L 13/17; D04H 1/4291; D04H 1/4382; D04H 3/16

(52) U.S. Cl. CPC A61L 2/18; A01N 25/10; A01N 25/34; A47L 13/17; D04H 1/4291; D04H 1/43838; D04H 3/16; A61L 2202/15; D10B 2321/022; D10B 2401/022; D10B 2401/04; D10B 2509/00

(57) ABSTRACT

Described herein is a nonwoven wipe comprising a plurality of polyolefin fibers, wherein the plurality of polyolefin fibers have an average fiber diameter of at least 200 nm and at most 3.5 micrometers, and wherein the plurality of polyolefin fibers have a basis weight of at least 20 and no more than 100 grams per square meter. Such nonwoven wipes can be used with an aqueous solution, optionally comprising an active ingredient, to decontaminate a surface. Also disclosed herein in a decontamination kit providing the same and a method of decontaminating a surface.

### TECHNICAL FIELD

A decontamination wipe made with a nonwoven having a small fiber diameter is described.

### SUMMARY

Microorganisms are known to be persistent on surfaces for extended periods of time. The combination of a decontaminating or cleaning solution and a wipe has been used for cleaning surfaces, such as an individuals' hands, counter tops, floors, and the like, that can potentially use both physical and chemical strategies. The wipes may be pre-wetted with the solution, and/or the solution may be added to the wipe right before or during use.

Disinfecting or sanitizing solutions are commonly used to kill microorganisms on contaminated surfaces. However, there are examples of microorganisms surviving the presence of a microbiocidal active agent, such as bacterial spores in the presence of quaternary ammonium compounds. Generally, decontamination wipes require a certain dwell time (i.e. the amount of time the surface needs to remain wet) to achieve disinfection. In some cases, dwell times can be as much as 10 minutes depending on the active ingredient.

Thus, there is a desire to identify new substrates that can be used in decontamination applications, which have improved decontaminating properties such as removal of hard to kill species, and/or a reduced dwell time. Additionally, or alternatively, there is a desire to identify wipes that are more durable especially when used in the presence of harsh chemicals.

In one aspect, a decontamination kit is described. The decontamination kit comprising:
(a) a nonwoven wipe comprising a plurality of polyolefin fibers, wherein the plurality of polyolefin fibers have an average actual fiber diameter of at least 200 nm and at most 3.5 micrometers, and wherein the plurality of polyolefin fibers have a basis weight of at least 20 and no more than 100 grams per square meter; and
(b) an aqueous solution, optionally comprising an active ingredient.

In another embodiment, a cleaning article is described comprising (a) a nonwoven wipe comprising a plurality of polyolefin fibers, wherein the plurality of polyolefin fibers have an average actual fiber diameter of at least 200 nm and at most 3.5 micrometers, and wherein the plurality of polyolefin fibers have a basis weight of at least 20 and no more than 100 grams per square meter; and (b) an aqueous solution, optionally comprising an active ingredient.

In yet another embodiment, a method of decontaminating a surface is described. The method comprising: contacting the surface with an aqueous solution, optionally comprising an active ingredient; and wiping the surface with a nonwoven wipe comprising a plurality of polyolefin fibers, wherein the plurality of polyolefin fibers have an average actual fiber diameter of at least 200 nm and at most 3.5 micrometers, and wherein the plurality of polyolefin fibers have a basis weight of at least 20 and no more than 100 grams per square meter.

The above summary is not intended to describe each embodiment. The details of one or more embodiments of the invention are also set forth in the description below. Other features, objects, and advantages will be apparent from the description and from the claims.

### DETAILED DESCRIPTION

As used herein, the term
“a”, “an”, and “the” are used interchangeably and mean one or more; and
“and/or” is used to indicate one or both stated cases may occur, for example A and/or B includes, (A and B) and (A or B),

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

As used herein, “comprises at least one of” A, B, and C refers to element A by itself, element B by itself, element C by itself, A and B, A and C, B and C, and a combination of all three.

It has been discovered that small diameter fiber nonwoven wipes can be more effective at decontamination of surfaces than larger diameter fiber nonwovens.

The wipes of the present disclosure are nonwoven. As used herein the term “nonwoven” generally refers to a fibrous web or material characterized by entanglement or point bonding of a plurality of fibers, wherein the fibers are interlaid, but not in an identifiable manner as in a knitted fabric.

The nonwoven wipes of the present disclosure comprise small diameter fibers. As disclosed herein, small diameter fibers refer to fibers having an actual average fiber diameter of at most 4.0, 3.5, 3.0, 2.5, 2.0, 1.5, 1.0, 0.9, 0.8, 0.7, 0.6, or even 0.5 micrometers. In one embodiment, the fibers have an actual average fiber diameter of at least 200, 300, 400, 500, 600, or even 700 nanometers (nm). The actual fiber diameter can be determined using techniques known in the art, for example, optical microscopy or scanning electron microscopy. In one embodiment, the fibers have an average effective fiber diameter of at most 8.0, 6.0, 5.0, 4.0, 3.5, or even 3.0 micrometers. In one embodiment, the fibers have an average effective fiber diameter of at least 1.0, 2.0, or even 2.5 micrometers. The term “Effective Fiber Diameter” means the apparent diameter of the fibers in a nonwoven fibrous web based on an air permeation test in which air at 1 atmosphere and room temperature is passed at a face velocity of 5.3 cm/sec through a web sample of known thickness, and the corresponding pressure drop is measured. Based on the measured pressure drop, the Effective Fiber Diameter is calculated as set forth in Davies, C. N., The Separation of Airborne Dust and Particles, Institution of Mechanical Engineers, vol. 167, issue 1b p. 185-213 (1953).

The small diameter fibers disclosed herein comprise a polyolefin-containing polymer.

Examples of such polyolefin-containing polymers may include, for instance, polyethylene, such as high density polyethylene (density of 0.94 to 0.97 g/cm³ as measured, for example, by ASTM D792-20), medium density polyethylene, low density polyethylene (density of 0.917 to 0.94 g/cm³), and linear low density polyethylene (density of 0.915 to 0.95 g/cm³); polypropylene, such as isotactic polypropylene, atactic polypropylene, and syndiotactic polypropylene; polybutylene, such as isotactic polybutylene, syndiotactic polybutylene, poly(1-butene) and poly(2-butene); polypentene, such as poly(1-pentene) and poly(2-pentene); poly(3-methyl-1-pentene); poly(4-methyl-1-pentene); and copolymers and blends thereof. Suitable copolymers include random and block copolymers prepared from two or more different unsaturated olefin monomers, such as ethylene/propylene and ethylene/butylene copolymers.

In one embodiment, the polyolefin polymer has a number average molecular weight (Mn) of at least 10,000; 20,000; 50,000; 80,000; or even 100,000 dalton, and at most 300,000; 500,000; 1,000,000; 2,000,000; or even 5,000,000 dalton as determined using techniques known in the art, such as gel permeation chromatography.

The polyolefin-containing polymer should be melt-processible and thus have some crystallinity.

In one embodiment, the crystalline polyolefin polymer has a moderate level of crystallinity, arising from stereoregular sequences in the polymer, for example stereoregular ethylene, propylene, or butylene sequences. For example, the polymer can be: (A) a propylene homopolymer in which the stereoregularity is disrupted in some manner such as by regio-inversions; (B) a random propylene copolymer in which the propylene stereoregularity is disrupted at least in part by co-monomers; or (C) a combination of (A) and (B). In a preferred exemplary embodiment, the polyolefin polymer is selected to be isotactic polypropylene, syndiotactic polypropylene, and mixtures thereof.

In some embodiments, the polyolefin polymer includes a non-conjugated diene monomer. The amount of diene present in the polymer is preferably less than 10% by weight, and more preferably less than 5% by weight. The diene may be any non-conjugated diene which is commonly used for the vulcanization of ethylene propylene rubbers including, but not limited to, ethylidene norbornene, vinyl norbornene, and dicyclopentadiene.

In another embodiment, the crystalline polyolefin polymer is a random copolymer of propylene and at least one co-monomer selected from ethylene, C₄-C₁₂alpha-olefins, and combinations thereof. In one particular embodiment, the copolymer includes ethylene-derived units in an amount ranging from at least 2, 5, 6, 8, or even 10% by weight and at most 20, 25, or even 28% by weight. This embodiment also includes propylene-derived units present in the copolymer in an amount ranging from at least 72, 75, or even 80% by weight to at most 98, 95, 94, 92, or even 90% by weight. These percentages by weight are based on the total weight of the propylene and ethylene-derived units; i.e., based on the sum of weight percent propylene-derived units and weight percent ethylene-derived units being 100%.

In yet another embodiment, the crystalline polyolefin polymer is a random propylene copolymer having a narrow compositional distribution. The copolymer is described as random because for a copolymer comprising propylene, co-monomer, and optionally diene, the number and distribution of co-monomer residues is consistent with the random statistical polymerization of the monomers. In stereoblock structures, the number of block monomer residues of any one kind adjacent to one another is greater than predicted from a statistical distribution in random copolymers with a similar composition. Historical ethylene-propylene copolymers with stereoblock structure have a distribution of ethylene residues consistent with these blocky structures rather than a random statistical distribution of the monomer residues in the polymer. The intramolecular composition distribution (i.e., randomness) of the copolymer may be determined by ¹³C NMR, which locates the co-monomer residues in relation to the neighboring propylene residues.

Exemplary commercially available polyolefins include those available from ExxonMobil Chemical Co. of Houston, Tex. under the trade designations “ACHIEVE” (propylene-based), “EXACT” (ethylene-based), and “EXCEED” (ethylene-based). Elastomeric olefin polymers are also commercially available from DuPont Dow Elastomers, LLC under the trade designation “ENGAGE” (ethylene-based); from Dow Chemical Co. of Midland, Mich. under the name “AFFINITY” (ethylene-based); from BASF, Florham Park, N.J., under the trade designation “STYROFLEX”; from Kraton Polymers, Houston, Tex., under the trade designation “PELLETHANE”, “INFUSE”, VERSIFY”, or “NORDEL”; from DSM, Heerlen, Netherlands, under the trade designation “ARNITEL”; from E. I. duPont de Nemours and Company, Wilmington, Del., under the trade designation “HYTREL”; and from ExxonMobil, Irving, Tex. under the trade designation “VISTAMAXX”.

In one embodiment, the polyolefin is an elastomeric block copolymer having the general formula A-B-A′ or A-B, wherein A and A′ are each a thermoplastic polymer endblock that contains a styrenic moiety and B is an elastomeric polymer midblock, such as a conjugated diene or a lower alkene polymer. Such copolymers may include, for instance, styrene-isoprene-styrene (S-I-S), styrene-butadiene-styrene (S-B-S), styrene-ethylene-butylene-styrene (S-EB-S), styrene-isoprene (S-I), styrene-butadiene (S-B), and so forth. Commercially available A-B-A′ and A-B-A-B copolymers include several different S-EB-S formulations from Kraton Polymers of Houston, Tex. under the trade designation “KRATON”. “KRATON” block copolymers are available in several different formulations, a number of which are identified in U.S. Pat. Nos. 4,663,220, 4,323,534, 4,834,738, 5,093,422 and 5,304,599, which are hereby incorporated by reference. Other commercially available block copolymers include the S-EP-S elastomeric polymers available from Kuraray Company, Ltd. of Okayama, Japan, under the trade designation “SEPTON”. Still other suitable polymers include the S-I-S and S-B-S elastomeric copolymers available from Dexco Polymers of Houston, Tex. under the trade designation “VECTOR”. Also suitable are polymers composed of an A-B-A-B tetrablock copolymer, such as discussed in U.S. Pat. No. 5,332,613 to Taylor, et al., which is incorporated herein by reference thereto. An example of such a tetrablock copolymer is a styrene-poly(ethylene-propylene)-styrene-poly(ethylene-propylene) (“S-EP-S-EP”) block copolymer. Examples of elastomeric polyolefin polymers are described in U.S. Pat. Nos. 5,278,272 and 5,272,236 to Lai, et al., U.S. Pat. No. 5,539,056 to Yang, et al., and U.S. Pat. No. 5,596,052 to Resconi, et al., which are incorporated herein by reference.

The crystallinity of the crystalline polyolefin polymers may be expressed in terms of heat of fusion. Embodiments of the present disclosure include crystalline polyolefin polymers exhibiting a heat of fusion as determined using differential scanning calorimetry (DSC) greater than 50, 51, 55, 60, 70, 80, 90, 100, or even about 110 J/g. Generally, the crystalline polyolefin polymers exhibit a heat of fusion as determined using DSC less than 210, 200, 190, 180, 170, 160, 150, 140, 130, 120, 110, or even less than 100 J/g.

The level of crystallinity is also reflected in the Melting Point. In one embodiment of the present disclosure, the polymer has a single Melting Point. Typically, a sample of propylene polymer will show secondary melting peaks adjacent to the principal peak, which are considered together as a single Melting Point. The highest of these peaks is considered to be the melting point. In one embodiment, the crystalline polyolefin polymer preferably has a melting point determined using DSC ranging from at most 300, 275, 250, 200, 175, 150, 125, 110, or even 105° C.; and at least 105, 110, 120, 125, 130, 140, 150, 160, 175, 180, 190, 200, 225, or even 250° C.

In one embodiment, the polyolefin-containing polymer is present in an amount of at least 50, 55, 60, 65, 70, 75, 80, 85, or even 90% weight in the nonwoven wipe. In one embodiment, the polyolefin-containing polymer is present in an amount of at least 100, 99, 98, 97, 96, 95, 90, 85, 80, 75, 70, 65, or even 60% by weight in the nonwoven wipe.

In some embodiments, the polyolefin-containing polymer may be blended with a tackifier to enable the smaller diameter fibers disclosed herein. Exemplary tackifiers include natural rosins and rosin esters, hydrogenated rosins and hydrogenated rosin esters, coumarone-indene resins, petroleum resins, polyterpene resins, and terpene-phenolic resins. Specific examples of suitable petroleum resins include, but are not limited to aliphatic hydrocarbon tackifier resins, hydrogenated aliphatic hydrocarbon tackifier resins, mixed aliphatic and aromatic hydrocarbon tackifier resins, hydrogenated mixed aliphatic and aromatic hydrocarbon tackifier resins, cycloaliphatic hydrocarbon tackifier resins, hydrogenated cycloaliphatic resins, mixed cycloaliphatic and aromatic hydrocarbon tackifier resins, hydrogenated mixed cycloaliphatic and aromatic hydrocarbon tackifier resins, aromatic hydrocarbon tackifier resins, substituted aromatic hydrocarbons, and hydrogenated aromatic hydrocarbon tackifier resins. In one embodiment the hydrocarbon tackifier is a saturated hydrocarbon, for example a C₅ piperylene derivative, and/or a C₉ resin oil derivative. Preferably, the tackifier is a hydrocarbon tackifier that is miscible (i.e., forms a homogenous melt) with the crystalline polyolefin polymer when the mixture is in a molten state, that is, when the mixture of the polyolefin polymer and the at least one hydrocarbon tackifier resin is heated to a temperature at or above the Melting Temperature (as determined using DSC) of the mixture. In one embodiment, the hydrocarbon tackifier resin comprises at least 2, 3, 4, 5, 7, 10, 15, or even 18% by weight and at most 40, 35, 30, 25, or even 20% by weight based on the weight of the nonwoven wipe.

In one embodiment, the small diameter fibers comprise from about 50% w/w to about 99% w/w of the polyolefin polymer, and from about 1% w/w to about 40% w/w of a hydrocarbon tackifier resin. In some embodiments, a single crystalline polyolefin polymer may be mixed with a single hydrocarbon tackifier resin. In other exemplary embodiments, a single crystalline polyolefin polymer may be advantageously mixed with two or more hydrocarbon tackifier resins. In further exemplary embodiments, two or more crystalline polyolefin polymers may be mixed with a single hydrocarbon tackifier resin. In other exemplary embodiments, two or more crystalline polyolefin polymers may be advantageously mixed with two or more hydrocarbon tackifier resins. Such embodiments are disclosed in U.S. Pat. Publ. No. 2020-0115833 (Joseph et al.), herein incorporated by reference.

In some embodiments, the polyolefin-containing polymer may be blended with other elastomeric materials, such as a polypropylene blended with a styrene block copolymer.

In some embodiments, the polyolefin-containing polymer may be blended with a plasticizer prior to fiber formation. In one embodiment, the amount of plasticizer is at least 0.001, 0.01, 0.1, 0.5, 0.75, or even 1% by weight and at most 30, 20, 10, 5, 2.5, or even 2.5% by weight based on the weight of the nonwoven wipe. In some such embodiments, the plasticizer is selected from oligomers of C₅ to C₁₄ olefins, and mixtures thereof. A non-limiting list of suitable commercially available plasticizers includes those plasticizers available under the trade designation “SHF” and “SUPEERSYN” available from Exxon-Mobil Chemical Company (Houston, TX); “STNFLUID” available from Chevron-Phillips Chemical Co. (Pasadena, TX); “DURASYN” available from BP-Amoco Chemicals (London, England); “NEXBASE” available from Fortum Oil and Gas Co. (Espoo, Finland); “SYNTON” available from Crompton Corporation (Middlebury, CT); and “EMERY” available from BASF GmbH (Ludwigshafen, Germany), formerly Cognis Corporation (Dayton, OH).

The nonwoven wipes containing small diameter fibers disclosed herein can be prepared as continuous fiber strands from processes as known in the art such as melt blowing processes, spun bonding processes, and solution spinning processes. In a melt blowing process, a nonwoven fibrous web is formed by extruding a fiber-forming material (e.g., the polyolefin-containing polymer, optional tackifier and optional additives) through one or more orifices to form filaments while contacting the filaments with air or other attenuating fluid to attenuate the filaments into discrete discontinuous fibers, and thereafter collecting a layer of the attenuated discrete discontinuous fibers. In a spunbound process, a molten fiber-forming material is extruded from a plurality of fine, usually circular, capillaries of a spinnerette with the diameter of the extruded fibers then being rapidly reduced as by, for example, eductive drawing and/or other well-known spunbonding mechanisms. The spunbound fibers are collected onto a surface forming a web. In solution spinning processes, the polymer is dissolved into a solvent and is extruded into a coagulation bath comprising another fluid that is compatible with the spinning solvent, but is not a solvent for the polymer or is extruded into a heated chamber of air and the solvent is evaporated. In another spinning process, an electric field is used to draw charged threads of liquid polymer as disclosed in US Pat. Publ. No. 2017/0137971 (Coffman). In yet another spinning process, liquid polymer is expelled from an orifice as the orifice is rotationally spun in a reservoir, which collects the polymeric fiber. Such a process of rotary jet spinning is disclosed in US Pat. Publ. No. 20150354094 (Parker et al.). These processes, which are known in the art, maybe used to make continuous small diameter fibers of the present disclosure.

Alternatively, short lengths of the small diameter fibers may be made or chopped from continuous strands and bonded together using secondary bonding processes known in the art to form the wipes of the present disclosure. Such bonding processes include the bonded carded process, through air bonding and pattern-roll bonding. In a bonded carded process, the small diameter fibers of the present disclosure are placed in a fiberizing unit/picker which separates the fibers. Next, the fibers are sent through a combining or carding unit which further breaks apart and aligns the staple fibers in the machine direction so as to form a machine direction-oriented fibrous non-woven web. Once the web has been formed, it is then bonded by one or more of several bonding methods. One bonding method is powder bonding wherein a powdered adhesive is distributed throughout the web and then activated, usually by heating the web and adhesive with hot air. Another bonding method is pattern bonding wherein heated calender rolls or ultrasonic bonding equipment is used to bond the fibers together, usually in a localized bond pattern through the web and or alternatively the web may be bonded across its entire surface if so desired. When using bi-component staple fibers, through-air bonding equipment is, for many applications, especially advantageous. Yet another bonding process includes a wet-laid process, which is analogous to a conventional papermaking process, where the small diameter fibers of the present disclosure along with optional other fibers and a binder are suspended in a fluid and the deposited onto a screen or porous surface to remove the fluid.

In yet another embodiment, the small diameter fibers may be made using a hydroentagling technique, wherein high-velocity water jets are used to wrap or knot individual fibers in a web bonding process. Such techniques are known in the art. See for example, U.S. Pat. Nos. 6,110,588, 5,207,970; and Pat. Publ. No. 20110250815.

Such processes disclosed above may be used to generate fibers having an actual fiber diameter greater than 3 microns. However, to make the very small diameter fibers, for example less than 3 micrometers, a tackifier optionally included. Such a process of adding a tackifer to make a small diameter fiber is disclosed in U.S. Pat. Publ. No. 2020-0115833 (Joseph et al.) and WO Publ. No. 2019025942 (Batra et al.) herein incorporated by reference.

In one embodiment, the nonwoven wipe is produced as a sheet or web which can be cut, die-cut or otherwise sized into the desired appropriate shape and size.

In one embodiment, the open-structured entangled mass of small diameter fibers (i.e., non-woven), which is typically in a web form is calendered by passing the nonwoven web through rollers, which are optionally heated, to obtain a compressed material.

After forming the nonwoven, the nonwoven may additionally or alternatively be wound into a storage roll for later processing if desired. Generally, once the nonwoven web has been collected, it may be conveyed to other apparatus such as a calender, embossing stations, laminators, cutters and the like; or it may be passed through drive rolls and wound into a storage roll. The nonwoven web may be cut into wipes for use in the present disclosure.

In one embodiment, the nonwoven wipe of the present disclosure has a basis weight of at least 20, 25, 30, 40, 50, or even 60 grams per square meter (gsm). If the basis weight is too low, there may not been enough contact between the fibers of the wipe and the microorganism on the surface. If the basis weight becomes too high, the cost of the wipe can increase. In one embodiment, the nonwoven wipe of the present disclosure has a basis weight of at most 120, 100, 75, 60, 55, or even 50 gsm.

The basis weight can be used to determine the solidity, which can be thought of as how solid (or dense) is a material in a particular volume. In one embodiment, the nonwoven wipe of the present disclosure has a solidity of at least 10, 12, 15, or even 20. In one embodiment, the nonwoven wipe of the present disclosure has a solidity of at most 40, 35, 30, 25, or even 20. If the solidity is too high, the material may act more as a film instead of a nonwoven material. As shown in the examples below, calendaring may be used to increase the solidity of a sample.

In some embodiments, the nonwoven wipes of the present disclosure may further comprise one or more optional components in addition to the polyolefin-containing small diameter fibers. The optional components may be used alone or in any combination suitable for the end-use application of the nonwoven wipes.

In one embodiment, the nonwoven wipe can, in addition to the small diameter fibers, also include larger diameter fibers, which may be the same or different composition from the small diameter fibers. In some embodiments, the additional fibers include polyester (e.g., polyethylene terephthalate), rayon, nylon (e.g., hexamethylene adipamide, polycaprolactam), acrylic (formed from a polymer of acrylonitrile), polypropylene, polyethylene, cellulose polymers, polyvinylidene chloride-vinyl chloride copolymers, vinyl chloride-acrylonitrile copolymers, and the like. Often, these additional fibers are staple fibers (i.e., a fiber that has been formed or cut to a staple length of generally 20 centimeters or less) blown into the web as the small diameter fibers are made.

In the present disclosure, the nonwoven wipes described herein have been discovered to work particularly well in containing microorganisms on the surface of a substrate.

The term “microorganism” is generally used to refer to any prokaryotic or eukaryotic microscopic organism, including without limitation, one or more of bacteria (e.g., motile or nonmotile, vegetative or dormant, Gram positive or Gram negative, planktonic or living in a biofilm), bacterial spores or endospores, algae, fungi (e.g., yeast, filamentous fungi, fungal spores), mycoplasmas, and protozoa, as well as combinations thereof. In some cases, the microorganisms of particular interest are those that are pathogenic, and the term “pathogen” is used to refer to any pathogenic microorganism. Examples of pathogens can include, but are not limited to, both Gram positive and Gram negative bacteria, fungi, and viruses including members of the family Enterobacteriaceae, or members of the family Micrococaceae, or the genera Staphylococcus spp., Streptococcus, spp., Pseudomonas spp., Acinetobacter spp., Enterococcus spp., Salmonella spp., Legionella spp., Shigella spp., Yersinia spp., Enterobacter spp., Escherichia spp., Bacillus spp., Listeria spp., Campylobacter spp., Acinetobacter spp., Vibrio spp., Clostridium spp., Klebsiella spp., Proteus spp. Aspergillus spp., and Candida spp. Viruses including lipid viruses such as the human immunodeficiency virus and the human respiratory syncytical virus; and non-lipid viruses such as polio virus, rhinovirus, norovirus, and Hepatitis A virus. Particular examples of pathogens can include, but are not limited to, Escherichia coli including enterohemorrhagic E. coli e.g., serotype O157:H7, O129:H11; Pseudomonas aeruginosa; Bacillus cereus; Bacillus anthracis; Salmonella enteritidis; Salmonella enterica serotype Typhimurium; Listeria monocytogenes; Clostridium botulinum; Clostridium perfringens; Staphylococcus aureus; methicillin-resistant Staphylococcus aureus; carbapenem-resistant Enterobacteriaceae, Campylobacter jejuni; Yersinia enterocolitica; Vibrio vulnificus; Clostridium difficile; vancomycin-resistant Enterococcus; Klebsiella pnuemoniae; Proteus mirabilus and Enterobacter [Cronobacter] sakazakii.

In the present application, the nonwoven wipe is used with an aqueous solution comprising an active ingredient to yield a decontamination wipe, which can be used to decontaminate a surface through cleaning, removal, sanitization, and/or disinfection.

The aqueous solution comprises water. The water can be tap water, distilled water, deionized water, and/or industrial soft water. In one embodiment, the water is deionized water and/or industrial soft water. The use of deionized water and/or industrial soft water reduces residue formation and limits the amount of undesirable metal ions in the aqueous solution. In one embodiment, the aqueous solution comprises at least 10, 20, 30, 40, or even 50 wt %. In one embodiment, water constitutes at least a majority weight percent of the aqueous solution (i.e, at least 50, 55, 70, 80, 90 or even 95 wt % of the aqueous solution).

In one embodiment, the wipes of the present disclosure remove the microorganisms from the surface by dislodging them.

In another embodiment, the wipes of the present disclosure kill the microorganisms on the substrate's surface through the use of an active ingredient (e.g., bactericidal, fungicidal, virucidal, tuberculocidal, sporicidal, etc.) in the aqueous solution which can kill specific microorganisms.

Exemplary active ingredients include: organic peracids, peroxides, quaternary ammonium-based compounds, chlorine-based compounds, surfactants, biguanides, alcohols, and those commonly used in the art.

Exemplary organic peracids include a peroxide derivative of one or more carboxylic acids. Suitable organic peracids may include, for instance, C1-C9 peracids, and particularly C1-C5 peracids. Examples of such peracids include performic acid, peracetic acid, perbenzoic acid, perpropionic acid, pemonanoic acid and halogen-substituted peracids, such as monochloroperacetic acid, dichloroperacetic acid, trichloroperacetic acid trifluoroperacetic acid, meta-chloroperoxybenzoic acid, as well as mixtures of the foregoing, and so forth.

Exemplary peroxides include hydrogen peroxide or another peroxide capable of releasing hydrogen peroxide when present in the solution. Suitable hydrogen peroxide sources may include, for example, peroxides of alkali and alkaline earth metals, organic peroxy compounds, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy complexes may include carbamide peroxide (also known as urea peroxide), alkyl and/or aryl peroxides (e.g., tert-butyl peroxide, diphenyl peroxide, etc.), alkyl and/or aryl ketone peroxides (e.g., benzyol peroxide), peroxy esters, diacyl peroxides, and mixtures thereof.

Exemplary quaternary ammonium-based compounds include dialkyl or alkyl benzyl quaternary ammonium chloride; dialkyl dimethylammonium compounds having either a carbonate or bicarbonate subgroup such as didecyl dimethylammonium carbonate/bicarbonate solution available from Lonza Inc. (Fair Lawn, N.J.) and sold under the trade designation of “CARBOQUAT 22C50”; dialkyl dimethlyammonium compound having sulfate groups, such as sulfate, methylsulfate, or ethylsulfate groups; and alkyl polyglucoside ammonium compounds. The alkyl polyglucoside ammonium compounds are derived from short to long alkyl chain sugars where the sugar or alkyl polyglucoside backbone is quaternized. An example of such a compound would be lauryldimethylammoniumhydroxypropyl alkyl polyglucosides such as sold by Colonial Chemical, Inc. (South Pittsburgh, Tenn.) under the trade designation of “SUGAQUAT” L-1010, L-1210, and L-8610. Quaternary ammonium-based compounds are well known in the art such as those disclosed in U.S. Pat. No. 8,859,481, herein incorporated by reference for its disclosure of quaternary ammonium-based compounds. Commercially available quaternary ammonium-chloride based aqueous solutions include alkyl ammonium halides such as lauryl trimethyl ammonium chloride and dilauryl dimethyl ammonium chloride; alkyl aryl ammonium halides such as octadecyl dimethyl benzyl ammonium bromide; ethyl dimethyl stearyl ammonium chloride, trimethyl stearyl ammonium chloride, trimethyl cetyl ammonium chloride, dimethyl ethyl lauryl ammonium chloride, dimethyl propyl myristyl ammonium chloride, dinonyl dimethyl ammonium chloride, didecyl dimethyl ammonium chloride, diundecyl dimethyl ammonium chloride, didecyl dimethyl ammonium chloride, dinonyly ethyl ammonium chloride, dimethyl ethyl benzyl ammonium chloride, 3-(trimethyxyosilyl) propyldidecylmethyl ammonium chloride, 3-(trimethoxysilyl) propyloctadecycdimethyl ammonium chloride, dimethyl dioctyl ammonium chloride, didecyl dimethyl ammonium chloride, didodecyl dimethyl ammonium chloride, dimethyl ditetradecyl ammonium chloride, dihekadecyl dimethyl ammonium chloride, dimethyl dioctadecyl ammonium chloride, decyl dimethyl octyl ammonium chloride, dimethyl dodecyloctyl ammonium chloride, benzyl decyl dimethyl ammonium chloride, benzyl dimethyl dodecyl ammonium chloride, benzyl dimethyl tetradecyl ammonium chloride, decyl dimethyl (ethyl benzyl) ammonium chloride, decyl dimethyl (dimethyl benzyl)-ammonium chloride, (chlorobenzyl)-decyl dimethyl ammonium chloride, decyl-(dichlorobenzyl)-dimethyl ammonium chloride, benzyl didecyl methyl ammonium chloride, benzyl didocyl methyl ammonium chloride, benzyl ditetradecyl methyl ammonium chloride, benzyl dodecyl ethyl methyl ammonium chloride, and the like. Some examples of commercially available quats include didecyl dimethyl ammonium chloride, available as BTC 1010 from Stepan Chemical Co.; “BARDAC 2250” from Lonza, Inc.; “FMB 210-15” from Huntington; “Maquat 4450-E” from Mason; dialkyl dimethyl ammonium chloride, available as BTC 818 from “BARDAC 2050”, Inc.; FMB 302 and Maquat 40 from Mason; and/or alkyl dimethyl benzyl ammonium chloride available as BTC 835 and “BARQUAT MB-50” from Lonza, Inc.; and FMB 451-5 and MC 1412 from Mason. Some quats are sold as mixtures of two or more different quats. Examples of these commercially available quat mixtures include, but are not limited to, twin chain blend/alkyl benzyl ammonium chloride compounds available under the trade designation “BARDAC 205M”, “BARDAC 208M”, and “BARQUAT 4250Z” from Lonza, Inc.; as BTC 885, BTC 888 and BTC 2250 from Stepan Chemical Co.; as FMB 504 and FMB 504-8 from Huntington; and as MQ 615M and MQ 624M from Mason. Further commercially available quaternary ammonium compounds include those sold under the trade designation “VIREX 11128 One-Step Disinfectant Cleaner and Deodorant” available from JohnsonDiversey, Inc. (Sturtevant, WI.); “5 L 3M Quat Disinfectant Cleaner” 5 L and 4 L 3M Bathroom Disinfectant Cleaner 4 L available from 3M Co., Maplewood, MN.

Exemplary chlorine-based compounds include: sodium hypochlorite bleach solutions, which are well known and are commonly available from many suppliers.

Surfactants can include nonionic, anionic, cationic, zwitterionic, and/or amphoteric surfactants. Many of these surfactants are described in in U.S. Pat. No. 6,673,761 (Mitra et al.) and U.S. Pat. No. 8,563,017 (Cuningham, et al.) and McCutcheon's Emulsifiers and Detergents (1997), Kirk-Othmer, Encyclopedia of Chemical Technology, 3rd Ed., Volume 22, pp. 332-432 (Marcel-Dekker, 1983), and McCutcheon's Soaps and Detergents (N. Amer. 1984), the contents of which are hereby incorporated by reference. Exemplary surfactants include: polysorbates (i.e., derived from ethoxylated sorbitan esterified with fatty acids), poloxomers (i.e., nonionic triblock copolymer composed of a central hydrophobic chain of polvoxypropylene (poly(propylene oxide)) flanked by two hydrophilic chains of polvoxvethylene (poly(ethylene oxide))), nonionic surfactants such as ethoxylated alcohols (e.g. commercially available from Evonik Industries under the trade designation “TOMADOL 25-7”), and amphoteric surfactant such as Octyl dimethyl amine oxide (e.g. commercially available from Lonza H&H under the trade designation Barlox 8S).

Commercially available examples of surfactants include alkyl glycosides available under the trade designation “GLUCOPON” 220, 225, 425, 600 and 625, all of which are available from Cognis Corp. of Cincinnati, OH; “TRITON” branded glycosides such as “TRITON” CG-110 and BG-10 available from Dow Chemical Co. of Midland, MI.

Exemplary biguanides include polyalkylene biguanides as disclosed in US Pat. Publ. No. 2014/0171512 (Kloeppel, et al.), herein incorporated by reference. An example of an polyalkylene biguanide is polyhexamethylene biguanide [also known as poly(iminoimidocarbonyliminoimidocarbonyliminohexamethylene) hydrochloride, or PHMB]; commercially available through Lonza Inc., Allendale, NJ, under the trade designation “VANTOCIL P—LONZA MICROBIOCIDE”.

Exemplary alcohols that may be used as an active ingredient include lower chain length alcohol such as ethanol or isopropanol. The inclusion of an alcohol and/or surfactant in the aqueous solution may act as a biocide, but it may also improve the cleaning performance of the decontamination wipe by improve wetting properties of the aqueous solution onto the nonwoven wipe, stabilizing the components in the aqueous solution, and/or function as an emulsifying agent depending on the nature of the alcohol and the surfactant.

The amount of the active ingredient in the aqueous solution can vary depending on the active ingredient used, whether or not is loaded onto the wipe, whether it is being used for a “germicidal” or “disinfectant” purpose, and/or where it is to be used. For example, sanitizers are safe for cleaning surfaces used in food preparation (e.g., restaurants and kitchens), while disinfectants are used to clean surfaces in hospital environments

In one embodiment, the amount of peracids in the aqueous solution is at least 0.01, 0.05, 0.1, or even 0.2 wt %. In one embodiment, the amount of peracids in the aqueous solution is at most 3, 2, 1, or even 0.5 wt %.

In one embodiment, the amount of peroxides in the aqueous solution is at least 0.5, 1, 2, or even 3 wt %. In one embodiment, the amount of peroxides in the aqueous solution is at most 15, 10, 8, 6, 5, 3, or even 2 wt %.

In one embodiment, the amount of quaternary ammonium compound in the aqueous solution is at least 0.05, 0.1, 0.2, 0.5, 0.75, or even 1 wt %. In one embodiment, the amount of quaternary ammonium in the aqueous solution is at most 5, 4, 3, 2, 1, or even 0.5 wt %.

In one embodiment, the amount of surfactant in the aqueous solution is at least 0.001, 0.002, 0.005, 0.01, 0.05, or even 0.1 wt %. In one embodiment, the amount of surfactant in the aqueous solution is at most 5, 4, 3, 2, 1, 0.5, 0.1, 0.05, or even 0.01 wt %.

In one embodiment, the amount of biguanide in the aqueous solution is at least 0.001, 0.002, 0.005, 0.01, 0.05, or even 0.1 wt %. In one embodiment, the amount of biguanide in the aqueous solution is at most 5, 4, 3, 2, 1, 0.5, 0.1, 0.05, or even 0.01 wt %.

In one embodiment, the amount of alcohol in the aqueous solution is at least 10, 20, 30, 40, or even 50 wt %. In one embodiment, the amount of alcohol in the aqueous solution is at most 90, 80, 70, 60, 50, 40, 30, 20, 10, or even 5 wt %.

Generally, the amount of active ingredient will decrease if the wipe is directed toward a sanitizing application versus a disinfectant application. For example, a sanitizer will only have 200-400 parts per million (ppm) of a quaternary ammonium-based compound in solution while a disinfectant will have about 600-3000 ppm of a quaternary ammonium compound in solution.

Optionally, the aqueous solution can include one or more additional components to enhance the functionality or aesthetics of the decontamination wipe. Additional components include, but are not limited to, buffering and pH adjusting agents, chelating agents, fragrances or perfumes, waxes, dyes and/or colorants, solubilizing materials, stabilizers, thickeners, defoamers, hydrotropes, lotions and/or mineral oils, enzymes, bleaching agents, cloud point modifiers, preservatives, and/or water-soluble polymers. Such additives are known in the art. See for example, U.S. Pat. No. 6,673,761 (Mitra et al.) and U.S. Pat. No. 8,563,017 (Cummingham et al.) herein incorporated by reference.

In one embodiment, a builder detergent is used to increase the effectiveness if a surfactant is present in the aqueous solution. The builder detergent can act as a softener and/or as a sequestering and buffering agent in the aqueous composition. Typically, the builder detergent includes sodium and/or potassium salts of ethylenediaminetetraacetic acid. The builder detergent content, when used in the aqueous solution, is typically about 0.01-0.8 weight percent.

In one embodiment, a solvent is used as a dispersion and solubilizing agent for the components of the aqueous solution, as a cleaning agent to help loosen and solubilize compounds, a residue inhibiting agent, an aid for wetting of the wipe, and/or a secondary disinfecting agent. Typically, the solvent is an alkanol such as methanol, ethanol, n-propanol, isopropanol, butanol, pentanol, and/or hexanol. In one embodiment, the amount of solvent in the aqueous solution is less than 10, 5, or even 1 weight percent of the aqueous solution.

In one embodiment, the polyolefin fiber is treated to aid the wetting of the wipe. In one embodiment, the polyolefin fiber is treated, for example, via plasma treatment or corona treatment to make the nonwoven wipe more hydrophilic in nature. In one embodiment, the nonwoven wipe of the present disclosure is substantially free (i.e., comprises less than 0.1%) of particles and coatings such as ionic polymer coatings.

The aqueous solution can be concentrated or unconcentrated. The aqueous solution may be incorporated into the wipe, or the aqueous solution may be added by the user to the wipe or substrate to be cleaned by the wipe.

The aqueous solution may be added to the wipers of the present disclosure by any method suitable for adding such solutions to substrates. The aqueous solution may be applied by any of the many well-known processes which include, but are not intended to, spraying, dipping, saturating, impregnating, brush coating, or other similar processes.

The aqueous solution is loaded onto the cleaning wipe to a desired loading ratio. In one embodiment, the aqueous solution is loaded to at least 100, 150, 200, 250, 300, or even 350% by weight versus the dry weight of the nonwoven wipe. In one embodiment, the aqueous solution is loaded to at most 600, 550, 500, 450, or even 400% by weight versus the dry weight of the nonwoven wipe. The loading of the decontamination wipe can be accomplished in several ways including, but not limited to, treating each individual wipe with a discrete amount of aqueous solution, mass treating a continuous web of cleaning wipes with the aqueous solution, soaking the entire web of cleaning wipes in the aqueous solution, spraying the aqueous solution in a stationary or moving web of cleaning wipes, and/or impregnating a stack of individually cut and sized cleaning wipes in a dispenser.

In another aspect of the present invention, the wipe containing the aqueous solution is individually sealed with a heat-sealable or glueable thermoplastic overwrap (such as polyethylene, Mylar and the like). In one embodiment, the decontamination wipes are packaged as numerous, individual sheets which are impregnated with the aqueous solution. In another embodiment, the nonwoven wipes are formed as a continuous web during the manufacturing process and loaded into a dispenser, such as a canister with a closure or a tub with closure. The closure is used to seal the nonwoven wipes loaded with the aqueous solution from the external environment and prevents premature volatilization of the components of the decontamination wipes. In one aspect of this embodiment, the dispenser includes a plastic such as, but not limited to, high density polyethylene, polypropylene, polycarbonate, polyethylene terephthalate (PET), polyvinyl chloride (PVC), and/or other rigid plastic. In another aspect of this embodiment, the continuous web of wipes is threaded through an opening in the top of the dispenser. In still another aspect of this embodiment, the dispenser includes a severing arrangement to cut a portion of the wipe after being removed from the dispenser. The severing arrangement can include, but is not limited to, a knife blade, serrated edge or the like. In still yet another aspect of this embodiment, the continuous web of wipes is scored, folded, segmented, and/or partially cut into uniform or non-uniform sizes and/or lengths. In a further aspect of this embodiment, the wipes are interleafed so that the removal of one wipe advances the next in the opening of the dispenser.

The decontamination wipe of the present invention may be used to disinfectant and/or sanitize any surface (e.g., food service counters, tables, medical instruments, high touch surfaces, bathroom counters, toilets, laboratory benches, bed rails, telephones, doorknobs, etc.).

In one embodiment, the decontamination wipe of the present invention may be more durable during use. For example, upon use, the wipe stays intact and does not rip, tear, etc.

Dwell time is how long a product must remain wet on the surface to kill disease-causing microorganisms. Dwell times can vary between products with common disinfectants taking up to 10 minutes to work depending on the organism. In one embodiment, the decontamination wipe of the present invention may enable reduced dwell times. For example, requiring a dwell time of no more than 15, 30, 45, or even 60 seconds.

Upon wiping, the decontamination wipe may provide a log reduction of at least 1.5, 2, 2.5, 3, 3.5, 4, or even 4.5 (e.g., a log reduction of 6). Log reduction, for example, may be determined from the % microbial population reduced by the decontamination wipe following the method disclosed in the Example Section.

Upon wiping, the decontamination wipe may provide a reduction of at least 25, 30, 35, 40, 45, or even 50% in the amount of microorganisms after cleaning. In some embodiments, the decontamination wipe may provide a reduction of at least 85, 90, 95, 98, or even 99% in the amount of microorganisms after cleaning.

### EXAMPLES

Unless otherwise noted, all parts, percentages, ratios, etc. in the examples and the rest of the specification are by weight, and all reagents used in the examples were obtained, or are available, from general chemical suppliers such as, for example, Sigma-Aldrich Company, Saint Louis, Missouri, or may be synthesized by conventional methods.

The following abbreviations are used herein: mL=milliliters, g=grams, lb=pounds, cm=centimeters, mm=millimeters, μm=micrometers, MHz=Mega Hertz, mTOrr=millitorr, sccm=standard cubic centimeters per minute, and wt %=percent by weight.

Methods

Fiber Diameter Measurement

Fiber diameter was determined using a Scanning Electron Microscope (SEM). The samples were sputter coated with gold in a vacuum chamber (Denton Vacuum, Moorestown, New Jersey). The specimens were then analyzed using a Phenom Pure SEM (Phenom-World, Eindhoven, Netherlands). The fiber diameter was reported as the average (mean) diameter determined from measurements taken on 500 individual fibers in the nonwoven web sample using SEM.

Effective Fiber Diameter Measurement

The Effective Fiber Diameter (EFD) of nonwoven webs was determined with an air flow rate of 32 L/minute (corresponding to a face velocity of 5.3 cm/second) using the method described in Davies, C. N., “he Separation of Airborne Dust and Particles,” Institution of Mechanical Engineers, London, Proceedings IB, 1952.

Solidity Measurement

Solidity (a) reported as a percentage was determined by the equation:

α=m_{f}÷(ρ_{f}×L_{nonwoven})×100%.

Basis weight, m_{f}, is the mass per surface area, ρ_{f} is the fiber density, and L_{nonwoven} is the nonwoven thickness.

Preparation of Nonwoven Webs for Wipes

Nonwoven Web A

The melt-blown (blown microfiber, BMF) nonwoven fibrous web was prepared using a crystalline polypropylene resin available under the trade designation METOCENE MF650Y resin (obtained from LyondellBasell, Houston, TX), having a melt flow rate (MFR) of 1800 g/10 min. A conventional melt-blowing process was employed similar to that described in Wente, Van A., “Superfine Thermoplastic Fibers” in Industrial Engineering Chemistry, Vol. 48, pages 1342 et seq. (1956). In particular, the melt-blowing die had circular smooth surfaced orifices, spaced 10 to the centimeter, with a 5:1 length to diameter ratio. Molten polymer was delivered to the die by a twin screw extruder. The extruder was equipped with two weight loss feeders to control the feeding of the polymer resin to the extruder barrel, and a gear pump to control the polymer melt flow to a die. The extruder temperature was about 250° C. and it delivered the melt stream to the BMF die, which was maintained at 250° C. The gear pump was adjusted so that a polymer throughput rate of 0.178 kg/hour/cm die width (1.0 lb/hour/inch die width) was maintained at the die. The primary air temperature of the air knives adjacent to the die orifices was maintained at about 350° C. The nonwoven web was produced on a rotating collector spaced 23 cm from the die with a collector speed of 7.6 meters/minute. The nonwoven web had an average fiber diameter of about 2.0 micrometers, an effective fiber diameter of 4.1 micrometers, a basis weight of 60 grams per square meter (gsm), and a solidity of 10.9%.

The web was then calendared using a smooth steel and a hexagon honeycomb pattern roll (sides of the patterned hexagons measured 3 mm) to produce the finished nonwoven web (solidity=11.5%).

Nonwoven Web B

The general procedure to form a nonwoven web as described for Nonwoven Web A was followed with the following differences. The polymer used was a blend at a 90/10 ratio by weight of METOCENE MF650Y resin and a hydrocarbon tackifier resin available under the trade designation OPPERA PR100A (obtained from the Exxon Mobil Corporation, Irving, TX). Molten polymer was delivered to the die by the twin screw extruder. The extruder was equipped with two weight loss feeders to control the feeding of the polymer resins to the extruder barrel, and a gear pump to control the polymer melt flow to a die. The extruder temperature was about 275° C. and it delivered the melt stream to the BMF die, which was maintained at 275° C. The gear pump was adjusted so that a polymer throughput rate of 0.178 kg/hour/cm die width (1.0 lb/hour/inch die width) was maintained at the die. The primary air temperature of the air knives adjacent to the die orifices was maintained at about 375° C. The nonwoven web was produced on a rotating collector spaced 23 cm from the die with a collector speed of 7.6 meters/minute. The nonwoven web had an average fiber diameter of about 1.6 micrometers, an effective fiber diameter of 3.2 micrometers, a basis weight of 57 gsm (grams per square meter), and a solidity of 12.6%.

The web was then calendared using a smooth steel and a hexagon honeycomb pattern roll (sides of the patterned hexagons measured 3 mm) to produce the finished nonwoven web (solidity=17.8%).

Nonwoven Web C

The general procedure to form a nonwoven web as described for Nonwoven Web A was followed with the following differences. The polymer used was a blend at an 85/15 ratio by weight of METOCENE MF650Y resin and the hydrocarbon tackifier resin OPPERA PR100A. The extruder temperature was about 340° C. and it delivered the melt stream to the BMF die, which was maintained at 325° C. The gear pump was adjusted so that a polymer throughput rate of 0.178 kg/hour/cm die width (1.0 lb/hour/inch die width) was maintained at the die. The primary air temperature of the air knives adjacent to the die orifices was maintained at about 400° C. The nonwoven web was produced on a rotating collector spaced 18 cm from the die with a collector speed of 6.4 meters/minute. The nonwoven web had an average fiber diameter of about 500 nm, an effective fiber diameter of 2.4 micrometers, a basis weight of 50 gsm, and a solidity of 11%.

The web was then calendared using a smooth steel and a hexagon honeycomb pattern roll (sides of the patterned hexagons measured 3 mm) to produce the finished nonwoven web (solidity=15.8%).

Nonwoven Web D

The same procedure to form a nonwoven web as described for Nonwoven Web C was followed with the exception that the web was produced on a rotating collector spaced 17 cm from the die with a collector speed of 12.8 meters/minute. The nonwoven web had an average fiber diameter of about 500 nm, an effective fiber diameter of 2.25 micrometers, a basis weight of 25 gsm, and a solidity of 11.5%.

The web was then calendared using a smooth steel and a hexagon honeycomb pattern roll (sides of the patterned hexagons measured 3 mm) to produce the finished nonwoven web (solidity=17.3%).

Nonwoven E

Nonwoven E was a commercial hydrophilic nonwoven wipe comprising polypropylene fibers available under the trade designation “06411 KIMTECH WETfASK Meltblown Sanitising Wipes” from the Kimberley-Clark Corporation).

Nonwoven F

Nonwoven F was a spunlaced, nonwoven sheet comprising polyester fibers available under the trade designation “SONTARA 8004” from the Jacob Holm Group, Basel, Switzerland.

The average fiber diameter for Nonwoven Webs A-D were determined following the Fiber Diameter Measurement Method described above. The basis weight was determined by weighing a known area of the sample (for example a 4 inch by 4 inch square). The average fiber diameter for Nonwovens E and F were determined by using a Keyence VK-200 confocal microscope with a 5× or 20× objective (Keyence Corporation).

TABLE 1
Average Fiber Dimeters and Basis Weights

| Nonwoven Web | Average Fiber Diameter | | Basis Weight (gsm) |
|---|---|---|---|
| A | 2.0 | micrometers | 60 |
| B | 1.6 | micrometers | 57 |
| C | 500 | nanometers | 50 |
| D | 500 | nanometers | 25 |
| E | 4.7 | micrometers | 34 |
| F | 11.5 | micrometers | 60 |

Plasma Treatment of Nonwoven Webs

A silicon containing film layer [methods of forming described in U.S. Pat. No. 6,696,157 (David) and U.S. Pat. No. 8,664,323 (Iyer) and US Pat. Appl. No. 2013/0229378 (Iyer)] was applied to a nonwoven sheet using a Plasma-Therm 3032 batch plasma reactor (obtained from Plasma-Therm LLC, St. Petersburg, FL). The instrument was configured for reactive ion etching with a 26 inch (66 centimeters) lower powered electrode and central gas pumping.

The chamber was pumped with a roots type blower (model EH1200 obtained from Edwards Engineering, Burgess Hill, UK) backed by a dry mechanical pump (model iQDP80 obtained from Edwards Engineering). The RF power was delivered by a 3 kW, 13.56 MHz solid-state generator (RFPP model RF30S obtained from Advanced Energy Industries, Fort Collins, CO). The system had a nominal base pressure of 5 mTorr. The flow rates of the gases were controlled by MKS flow controllers (obtained from MKS Instruments, Andover, MA).

Nonwoven web samples were fixed on the powered electrode of the plasma reactor. After pumping down to the base pressure, the gases tetramethylsilane (TMS) and oxygen (O₂) were introduced at flow rates of 150 sccm and 500 sccm, respectively. Once the gas flows stabilized in the reactor, rf (radio frequency) power (1000 watts) was applied to the electrode to generate the plasma. The plasma exposure time was 30 seconds. Following completion of the first plasma treatment, the sample was exposed to oxygen once more and rf power (1000 watts) was applied to the electrode to generate the plasma for 20 seconds. Following completion of the plasma treatment, the chamber was vented to the atmosphere and the treated nonwoven sample was removed from the chamber.

Preparation of Inoculum for Test Plates

The inoculum stock solution was prepared by combining a 1 mL aliquot of Clostridium sporogenes (ATCC 3584) spores (about 1×10⁸ spores/mL of water) with sterile water (8.5 mL) and 0.5 mL of fetal bovine serum (obtained from Thermo Fisher Scientific, Waltham, MA). The inoculum stock solution was chilled with ice until used.

Preparation of Inoculated Test Plates

Stainless-steel (304 grade) test plates (12.7 cm×18 cm) were rinsed with distilled water and then sprayed with a 10% by volume bleach solution. The bleach solution was maintained on the plates for 5 minutes followed by a thorough rinsing of the plates with distilled water. The plates were then sprayed with a 70% by volume aqueous solution of ethanol or isopropanol. The plates were dried and then autoclaved for a minimum of 20 minutes at 121° C. The autoclaved plates were equilibrated to room temperature, washed with sterile water, and then individually wiped with clean wipes available under the trade designation KIMWIPE (obtained from the Kimberley-Clark Corporation, Irving, TX). This was followed by spraying the plates with a 70% aqueous solution of ethanol or isopropanol and then individually drying the plates with clean KIMWIPE wipers.

An aliquot (100 microliters) of inoculum solution was added to the center of each plate and spread out to cover a 2.5 cm by 5 cm area. A polyvinyl chloride (PVC) dowel was used as the inoculum spreader. Prior to use each dowel was subjected to a pre-treatment process. In the pre-treatment process, each dowel was immersed in a 10% by volume aqueous bleach solution for 5 minutes. Each dowel was then sequentially immersed in sterile water for 30 seconds, immersed in a 70% aqueous solution of ethanol or isopropanol for 30 seconds, and air dried.

Procedure for Decontaminating a Surface with a Wipe

A mechanical test apparatus to measure the decontamination of a solid surface using a nonwoven wipe was assembled as described in U.S. Pat. No. 10,087,405 (Swanson). The apparatus included an orbital shaker (hermolyne Roto-Mix orbital shaker Model Type 50800) with a lever arm for holding a wipe attached to one edge of the shaker table. The lever arm was a 7.5 cm by 16.5 cm by 1.3 cm steel plate that was attached to the shaker table with a hinged mounting. For each test, a single test wipe was wrapped as a flat sheet around the arm and secured in place so that a 7.5 cm by 10.2 cm section of one surface of the arm was covered with the wipe. An inoculated test plate was placed on a table next to the test apparatus. In operation, the hinged arm was lowered from a vertical (i.e. non-operational) position to the horizontal operational position in which the wipe covered surface of the arm was laid flat on top of and in contact with the test plate. The arm and test plate were oriented so that the wipe was centered over the inoculated portion of the plate. The weight of the arm on the test plate was about 350 g. The inoculated area of the test plate was wiped with the nonwoven wipe by operating the orbital shaker at a setting of 100 rotations/minute for 15 seconds. Following the wiping procedure, the arm was removed from the surface of the test plate by returning it to the vertical position. Control plates for the procedure were also prepared in which the plates were inoculated, but not wiped with a test wipe.

C. sporogenes spores that remained on the test plate following the wiping procedure were recovered after the surface dried by wiping the surface of the plate with a polyurethane foam swab (TX712A CLEANFOAM swab, 13 mm×25.7 mm foam swab head, obtained from Texwipe, Kemersville, NC) that had been pre-wetted by soaking the swab in a 0.05% aqueous polysorbate 20 solution (Alfa Aesar, Tewksbury, MA). The plate surface was swabbed with the swab using the following 3-step procedure: 1) swabbed twice in a diagonal direction (back and forth, switching sides of the swab between each direction); 2) swabbed twice in the lengthwise direction (back and forth, switching sides of the swab between each direction); and 3) swabbed twice in the widthwise direction (back and forth, switching sides of the swab between each direction). C. sporogenes spores remaining on the control plates were recovered using the same procedure.

The foam section of each swab was cut from the handle portion using sterilized scissors and then immersed into a tube containing 10 mL of Letheen broth (BD DIFCO brand, Becton Dickinson, Franklin Lakes, NJ). The contents were mixed by placing the tube in an ultrasonic bath sonicator at room temperature for one minute. After sonication, the contents were mixed for one minute at room temperature using a vortex mixer. An aliquot of liquid (1 mL) was removed from the tube and serially diluted (10-fold dilutions) using Butterfield's buffer (obtained from the 3M Company, Maplewood, MN) to yield a C. sporogenes concentration level that provided counts of colony forming units (cfu) within the counting range of a 3M PETRIFILM Aerobic Count Plate (3M Company). An aliquot (1 mL) from each diluted sample was plated on separate petrifilm plates available under the trade designation 3M PETRIFILM Aerobic Count Plate according to the manufacturer's instructions. The count plates were incubated at 37° C. for 20-24 hours in an anaerobic chamber. After the incubation period, the number of cfu on each count plate were counted by visual examination. The count value was used to calculate the total number of cfu recovered from a test plate or control plate. The results were reported as the mean log₁₀ cfu count from 3 or 4 trials.

Aqueous Solution A

Aqueous Solution A was prepared by diluting a cleaning concentrate available under the trade designation “3M Disinfectant Cleaner RCT Concentrate 40” (active ingredients: octyl decyl dimethyl ammonium chloride, dioctyl dimethyl ammonium chloride, dodecyl dimethyl ammonium chloride, and alkyl (C14, C12 and C16) dimethyl benzyl ammonium chloride; obtained from the 3M Company) with water at a volume ratio of 1:256.

Aqueous Solution B

Aqueous Solution B was prepared by diluting a cleaning concentrate available under the trade designation “3M Neutral Quat Disinfectant Cleaner Concentrate” (active ingredients: octyl decyl dimethyl ammonium chloride, dioctyl dimethyl ammonium chloride, didecyl dimethyl ammonium chloride, and alkyl (C14, C12 and C16) dimethyl benzyl ammonium chloride; obtained from the 3M Company) in water at a ratio of 1:256.

Aqueous Solution C

A 0.1 wt % aqueous solution was prepared of a nonionic, polysorbate surfactant available under the trade designation “TWEEN 20” (obtained from Alfa Aesar).

### Example 1

A 10.2 cm by 15.2 cm sample of Nonwoven Web D was used as the test wipe.

The test wipe was weighed and then placed flat in a re-sealable plastic bag. Aqueous Solution A, in an amount 4× the weight of the test wipe, was added to the plastic bag and the bag was sealed. A hand-roller was used external to the bag to incorporate the disinfectant solution into the wipe. The finished wipe was maintained in the sealed bag until evaluated using the ‘Procedure for Decontaminating a Surface with a Wipe’ described above. The mean log₁₀ cfu count (n=3) is reported in Table 2 together with the calculated log₁₀ cfu reduction achieved by wiping the inoculated test plate with the nonwoven wipe.

### Example 2

The same procedures for preparing and evaluating a test wipe as described in Example 1 were followed with the exception that a sample of Nonwoven Web C was used as the test wipe. The mean log₁₀ cfu count (n=3) is reported in Table 2 together with the calculated log₁₀ cfu reduction achieved by wiping the inoculated test plate with the nonwoven wipe.

### Comparative Example A

The same procedures for preparing and evaluating a test wipe as described in Example 1 were followed with the exception that Nonwoven E was used as the test wipe. The mean log₁₀ cfu count (n=3) is reported in Table 2 together with the calculated log₁₀ cfu reduction achieved by wiping the inoculated test plate with the nonwoven wipe.

### Example 3

A 10.2 cm by 15.2 cm sample of Nonwoven Web C was used as the test wipe. The test wipe was weighed and then placed flat in a re-sealable plastic bag. Disinfectant Solution B, in an amount 4× the weight of the test wipe, was added to the plastic bag and the bag was sealed. A hand-roller was used external to the bag to incorporate the disinfectant solution into the wipe. The finished wipe was maintained in the sealed bag until evaluated using the ‘Procedure for Decontaminating a Surface with a Wipe’ described above. The mean log₁₀ cfu count (n=3) is reported in Table 2 together with the calculated log₁₀ cfu reduction achieved by wiping the inoculated test plate with the nonwoven wipe.

### Comparative Example B

The same procedures for preparing and evaluating a test wipe as described in Example 3 were followed with the exception that Nonwoven E was used as the test wipe. The mean log₁₀ cfu count (n=3) is reported in Table 2 together with the calculated log₁₀ cfu reduction achieved by wiping the inoculated test plate with the nonwoven wipe.

TABLE 2
Log₁₀ CFU Reduction Results

| | | | Mean Log₁₀ CFU Recovered (C. sporogenes) | | |
|---|---|---|---|---|---|
| | | | from a Test Plate (n = 3), | | |
| | | | SD = Standard Deviation | | |
| | | | Plate Not Wiped | Plate Wiped | Log₁₀ CFU |
| | | Aqueous | with Test Wipe | with Test Wipe | Reduction |
| Test Wipe | Nonwoven | Solution | (Control Plate) | (Test Plate) | from Wiping |
| Example 1 | D | A | 5.93 (SD = 0.04) | 1.36 (SD = 0.32) | 4.57 |
| Example 2 | C | A | 5.93 (SD = 0.04) | 1.80 (SD = 0.61) | 4.13 |
| Comparative | E | A | 5.93 (SD = 0.04) | 2.47 (SD = 0.16) | 3.46 |
| Example A | | | | | |
| Example 3 | C | B | 5.37 (SD = 0.06) | 1.0 (SD = 0.03) | 4.37 |
| Comparative | E | B | 5.37 (SD = 0.06) | 1.6 (SD = 0.38) | 3.77 |
| Example B | | | | | |

Spores, such as C. sporogenes, a non pathogenic pore, are known to not be neutralized or killed with typical quaternary ammonium compounds such as those found in Aqueous Solutions A and B. Thus, the reduction in C. sporogenes in the above Table is believed to be due to the physical removal of the spores from the surface using the wipe.

### Example 4

A 10.2 cm by 15.2 cm sample of Nonwoven Web B was used as the test wipe. The test wipe was weighed and then placed flat in a re-sealable plastic bag. Aqueous Solution C in an amount 4× the weight of the test wipe, was added to the plastic bag and the bag was sealed. A hand-roller was used external to the bag to incorporate the surfactant solution into the wipe. The finished wipe was maintained in the sealed bag until evaluated using the ‘Procedure for Decontaminating a Surface with a Wipe’ described above. The mean log₁₀ cfu count (n=4) is reported in Table 3 together with the calculated log₁₀ cfu reduction achieved by wiping the inoculated test plate with the nonwoven wipe.

### Example 5

The same procedures for preparing and evaluating a test wipe as described in Example 4 were followed with the exception that the Nonwoven Web B sample was plasma treated (as described in the procedure above) before Aqueous Solution C was incorporated. The mean log₁₀ cfu count (n=4) is reported in Table 3 together with the calculated log₁₀ cfu reduction achieved by wiping the inoculated test plate with the nonwoven wipe.

TABLE 3
Log₁₀ CFU Reduction Results

| | | Mean Log₁₀ CFU Recovered (C. sporogenes) | | |
|---|---|---|---|---|
| | | from a Test Plate (n = 4), | | |
| | | SD = Standard Deviation | | |
| | | Plate Not Wiped | Plate Wiped | Log₁₀ CFU |
| | | with Test Wipe | with Test Wipe | Reduction |
| Test Wipe | Nonwoven | (Control Plate) | (Test Plate) | from Wiping |
| Example 4 | B | 5.64 (SD = 0.21) | 3.86 (SD = 0.24) | 1.78 |
| Example 5 | B (Plasma | 5.64 (SD = 0.21) | 2.51 (SD = 0.13) | 3.13 |
| | treated) | | | |

### Example 6

A 10.2 cm by 15.2 cm sample of Nonwoven Web C that had been plasma treated (as described in the procedure above) was used as the test wipe. The test wipe was weighed and then placed flat in a re-sealable plastic bag. Aqueous Solution C, in an amount 4× the weight of the test wipe, was added to the plastic bag and the bag was sealed. A hand-roller was used external to the bag to incorporate the surfactant solution into the wipe. The finished wipe was maintained in the sealed bag until evaluated using the ‘Procedure for Decontaminating a Surface with a Wipe’ described above. The mean log₁₀ cfu count (n=3) is reported in Table 4 together with the calculated log₁₀ cfu reduction achieved by wiping the inoculated test plate with the nonwoven wipe.

### Example 7

The same procedures for preparing and evaluating a test wipe as described in Example 6 were followed with the exception that a sample of plasma treated Nonwoven Web A was used as the test wipe. The mean log₁₀ cfu count (n=3) is reported in Table 4 together with the calculated log₁₀ cfu reduction achieved by wiping the inoculated test plate with the nonwoven wipe.

### Comparative Example C

The same procedures for preparing and evaluating a test wipe as described in Example 6 were followed with the exception that Nonwoven F was used as the test wipe. The mean log₁₀ cfu counts (n=3) are reported in Table 4 together with the calculated log₁₀ cfu reduction achieved by wiping the inoculated test plate with the nonwoven wipe.

### Comparative Example D

The same procedures for preparing and evaluating a test wipe as described in Example 6 were followed with the exception that a KIMTECH WETTASK #06411 sanitizing wipe was used as the test wipe. The mean log₁₀ cfu count (n=3) is reported in Table 4 together with the calculated log₁₀ cfu reduction achieved by wiping the inoculated test plate with the nonwoven wipe.

TABLE 4
Log₁₀ CFU Reduction Results

| | | Mean Log₁₀ CFU Recovered (C. sporogenes) | | |
|---|---|---|---|---|
| | | from a Test Plate (n = 3), | | |
| | | SD = Standard Deviation | | |
| | | Plate Not Wiped | Plate Wiped | Log₁₀ CFU |
| | | with Test Wipe | with Test Wipe | Reduction |
| Test Wipe | Nonwoven | (Control Plate) | (Test Plate) | from Wiping |
| Example 6 | C (plasma | 5.69 (SD = 0.10) | 2.38 (SD = 0.49) | 3.31 |
| | treated) | | | |
| Example 7 | A (plasma | 5.69 (SD = 0.10) | 1.86 (SD = 0.13) | 3.83 |
| | treated) | | | |
| Comparative | F | 5.69 (SD = 0.10) | 3.91 (SD = 0.13) | 1.78 |
| Example C | | | | |
| Comparative | E | 5.69 (SD = 0.10) | 2.52 (SD = 0.10) | 3.17 |
| Example D | | | | |

Similar to Table 2, C. sporogenes spores, are known to not be neutralized or killed with the surfactant compound found in Aqueous Solution C. Thus, the reduction in C. sporogenes in the above Table is believed to be due to the physical removal of the spores from the surface using the wipe.

Foreseeable modifications and alterations of this invention will be apparent to those skilled in the art without departing from the scope and spirit of this invention. This invention should not be restricted to the embodiments that are set forth in this application for illustrative purposes. To the extent that there is any conflict or discrepancy between this specification as written and the disclosure in any document mentioned or incorporated by reference herein, this specification as written will prevail.

1. A decontamination kit comprising:
(a) a nonwoven wipe comprising a plurality of polyolefin fibers, wherein the plurality of polyolefin fibers have an average actual fiber diameter of at least 200 nm and at most 3.5 micrometers, and wherein the plurality of polyolefin fibers have a basis weight of at least 20 and no more than 100 grams per square meter; and
(b) an aqueous solution.

2. The kit of claim 1, wherein the aqueous solution comprises an active ingredient.

3. The kit of claim 2, wherein the active ingredient comprises at least one of an organic peracid, peroxide, quaternary ammonium-based compound, chlorine-based compound, surfactant, biguanide, and alcohol.

4. The kit of claim 1, wherein the plurality of polyolefin fibers comprises at least one of polypropylene and polyethylene.

5. The kit of claim 1, wherein the plurality of polyolefin fibers comprises from about 50% to about 99% by weight of at least one crystalline polyolefin polymer, and from about 1% to about 40% by weight of at least one hydrocarbon tackifier resin, wherein the nonwoven wipe exhibits a Heat of Fusion measured using Differential Scanning Calorimetry of greater than 50 Joules/gram.

6. The kit of claim 1, wherein the plurality of polyolefin fibers has a basis weight of at least 20 and no more than 60 grams per square meter.

7. The kit of claim 1, wherein the plurality of polyolefin fibers has an average actual fiber diameter of at least 200 nm and at most 900 nm.

8. The kit of claim 1, wherein the plurality of polyolefin fibers comprise a hydrocarbon tackifier.

9. The kit of claim 8, wherein the hydrocarbon tackifier is a saturated hydrocarbon.

10. The kit of claim 1, wherein the kit can provide a reduction in microorganisms of at least 90% from a surface after use.

11. The kit of claim 1, wherein the kit provides a reduction in microorganisms of at least 99% from a surface after use.

12. The kit of claim 1, wherein the plurality of polyolefin fibers is substantially free of a coating.

13. The kit of claim 1, wherein the aqueous solution is provided on the nonwoven wipe.

14. The kit of claim 1, wherein the aqueous solution is not provided on the nonwoven wipe.

15. The kit of claim 1, wherein the plurality of polyolefin fibers are treated to impart hydrophilicity.

16. The kit of claim 15, wherein the aqueous solution comprises a solvent or a surfactant.

17. A moist towelette comprising (a) a nonwoven wipe comprising a plurality of polyolefin fibers, wherein the plurality of polyolefin fibers have an average actual fiber diameter of at least 200 nm and at most 3.5 micrometers, and wherein the plurality of polyolefin fibers have a basis weight of at least 20 and no more than 100 grams per square meter; and (b) an aqueous solution.

18. A method of decontaminating a surface, the method comprising:
contacting the surface with an aqueous solution; and wiping the surface with a nonwoven wipe comprising a plurality of polyolefin fibers, wherein the plurality of polyolefin fibers have an average fiber diameter of at least 200 nm and at most 3.5 micrometers, and wherein the plurality of polyolefin fibers have a basis weight of at least 20 and no more than 100 grams per square meter.

19. The kit of claim 1, wherein the aqueous solution comprises at least 50 wt % of water.

20. The kit of claim 1, wherein the aqueous solution comprises at least 95 wt % of water.
